(19) United States
(12) Patent Application Publication
(10) Pub. No.: US 2006/0018804 A1
(43) Pub. Date: Jan. 26, 2006

(54) Enhanced germicidal lamp

(75) Inventor: Gregory S. Snyder, San Francisco, CA (US)

Correspondence Address: BELL, BOYD & LLOYD LLC, P.O. BOX 1135, CHICAGO, IL, 60690-1135, US

(73) Assignee: Sharper Image Corporation, San Francisco, CA (US)

(21) Appl. No.: 11/004,397

(22) Filed: Dec. 3, 2004

Related U.S. Application Data
(60) Provisional application No. 60/590,445, filed on Jul. 23, 2004.

(51) Int. Cl. A61L 9/20

(52) U.S. Cl. 422/121

(57) ABSTRACT

An integrally shielded germicidal lamp to selectively direct UV light emitted preferably within an air transporting and/or conditioning system. The system comprises a housing, an emitter electrode configured within the housing, a collector electrode configured within the housing and positioned downstream from the emitter electrode, and a therefrom. The system preferably includes a driver electrode is preferably removable from the housing through a side portion of the housing. Preferably, the driver electrode is insulated with a dielectric material and/or a catalyst. Preferably, a removable trailing electrode is configured within the housing and downstream of the collector electrode. Preferably, a first voltage source electrically is coupled to the emitter electrode and the collector electrode, and a second voltage source electrically is coupled to the trailing electrode. The second voltage source is independently and selectively controllable of the first voltage source.

### CLAIM OF PRIORITY

The present application claims priority under 35 USC 119(e) to U.S. Patent Application No. 60/590,445, filed Jul. 23, 2004, entitled “Air Conditioner Device With Enhanced Germicidal Lamp” (Attorney Docket No. SHPR-01361USR), which is hereby incorporated by reference.

### CROSS-REFERENCE APPLICATION

The present invention is related to the following patent applications and patents, each of which is incorporated herein by reference:

U.S. patent application Ser. No. 10/074,207, filed Feb. 12, 2002, entitled “Electro-Kinetic Air Transporter-Conditioner Devices with Interstitial Electrode” (Attorney Docket No. SHPR-01041USN);

U.S. Pat. No. 6,176,977, entitled “Electro-Kinetic Air Transporter-Conditioner” (Attorney Docket No. SHPR-01041US0);

U.S. Pat. No. 6,544,485, entitled “Electro-Kinetic Device with Anti Microorganism Capability” (Attorney Docket No. SHPR-01028US0);

U.S. patent application Ser. No. 10/074,347, filed Feb. 12, 2002, and entitled “Electro-Kinetic Air Transporter-Conditioner Device with Enhanced Housing” (Attorney Docket No. SHPR-01028US5);

U.S. patent application Ser. No. 10/717,420, filed Nov. 19, 2003, entitled “Electro-Kinetic Air Transporter And Conditioner Devices With Insulated Driver Electrodes” (Attorney Docket No. SHPR-01414US1);

U.S. patent application Ser. No. 10/625,401, filed Jul. 23, 2003, entitled “Electro-Kinetic Air Transporter And Conditioner Devices With Enhanced Arcing Detection And Suppression Features” (Attorney Docket No. SHPR-01361USB);

U.S. Pat. No. 6,350,417 issued May 4, 2000, entitled “Electrode Self Cleaning Mechanism For Electro-Kinetic Air Transporter-Conditioner” (Attorney Docket No. SHPR-01041US1);

U.S. Pat. No. 6,709,484, issued Mar. 23, 2004, entitled “Electrode Self-Cleaning Mechanism For Electro-Kinetic Air Transporter Conditioner Devices (Attorney Docket No. SHPR-01041US5);

U.S. Pat. No. 6,350,417 issued May 4, 2000, and entitled “Electrode Self Cleaning Mechanism For Electro-Kinetic Air Transporter-Conditioner” (Attorney Docket No. SHPR-01041US1);

U.S. patent application Ser. No. 60/590,688, filed Jul. 23, 2004, entitled “Air Conditioner Device With Removable Driver Electrodes” (Attorney Docket No. SHPR-01361USA);

U.S. patent application Ser. No. 60/590,735, filed Jul. 23, 2003, entitled “Air Conditioner Device With Variable Voltage Controlled Trailing Electrodes” (Attorney Docket No. SHPR-01361USG);

U.S. patent application Ser. No. 60/590,960, filed Jul. 23, 2003, entitled “Air Conditioner Device With Removable Interstitial Driver Electrodes” (Attorney Docket No. SHPR-01361USQ);

U.S. patent application Ser. No. ______, filed ______, entitled “Air Conditioner Device With Removable Driver Electrodes” (Attorney Docket No. SHPR-01414US7);

U.S. patent application Ser. No. ______, filed ______, entitled “Air Conditioner Device With Variable Voltage Controlled Trailing Electrodes” (Attorney Docket No. SHPR-01414US8);

U.S. patent application Ser. No. ______, filed ______, entitled “Air Conditioner Device With Individually Removable Driver Electrodes” (Attorney Docket No. SHPR-01414US9);

U.S. patent application Ser. No. ______, filed ______, entitled “Air Conditioner Device With Enhanced Germicidal Lamp” (Attorney Docket No. SHPR-01414USA); and

U.S. patent application Ser. No. ______, filed ______, entitled “Air Conditioner Device With Removable Driver Electrodes” (Attorney Docket No. SHPR-01414USB).

### FIELD OF THE INVENTION

The present invention is related generally to a system for conditioning and/or transporting air.

### BACKGROUND OF THE INVENTION

The use of an electric motor to rotate a fan blade to create an airflow has long been known in the art. Although such fans can produce substantial airflow (e.g., 1,000 ft³/minute or more), substantial electrical power is required to operate the motor, and essentially no conditioning of the flowing air occurs.

It is known to provide such fans with a HEPA-compliant filter element to remove particulate matter larger than perhaps 0.3 μm. Unfortunately, the resistance to airflow presented by the filter element may require doubling the electric motor size to maintain a desired level of airflow. Further, HEPA-compliant filter elements are expensive, and can represent a substantial portion of the sale price of a HEPA-compliant filter-fan unit. While such filter-fan units can condition the air by removing large particles, particulate matter small enough to pass through the filter element is not removed, including bacteria, for example.

It is also known in the art to produce an airflow using electro-kinetic techniques whereby electrical power is converted into a flow of air without utilizing mechanically moving components. One such system is described in U.S. Pat. No. 4,789,801 to Lee (1988), depicted herein in simplified form as FIGS. 1A and 1B, which is hereby incorporated by reference. System 10 includes an array of first (“emitter”) electrodes or conductive surfaces 20 that are spaced-apart from an array of second (“collector”) electrodes or conductive surfaces 30. The positive terminal of a generator such as, for example, pulse generator 40 which outputs a train of high voltage pulses (e.g., 0 to perhaps +5 KV) is coupled to the first array 20, and the negative pulse generator terminal is coupled to the second array 30 in this example.

The high voltage pulses ionize the air between the arrays 20, 30 and create an airflow 50 from the first array 20 toward the second array 30, without requiring any moving parts. Particulate matter 60 entrained within the airflow 50 also moves towards the second electrodes 30. Much of the particulate matter is electrostatically attracted to the surfaces of the second electrodes 30, where it remains, thus conditioning the flow of air that is exiting the system 10. Further, the high voltage field present between the electrode sets releases ozone O₃, into the ambient environment, which eliminates odors that are entrained in the airflow.

In the particular embodiment of FIG. 1A, the first electrodes 20 are circular in cross-section, having a diameter of about 0.003″ (0.08 mm), whereas the second electrodes 30 are substantially larger in area and define a “teardrop” shape in cross-section. The ratio of cross-sectional radii of curvature between the bulbous front nose of the second electrode 30 and the first electrodes 20 exceeds 10:1. As shown in FIG. 1A, the bulbous front surfaces of the second electrodes 30 face the first electrodes 20, and the somewhat “sharp” trailing edges face the exit direction of the airflow. In another particular embodiment shown herein as FIG. 1B, second electrodes 30 are elongated in cross-section. The elongated trailing edges on the second electrodes 30 provide increased area upon which particulate matter 60 entrained in the airflow can attach.

### BRIEF DESCRIPTION OF THE FIGURES

FIG. 1A illustrates a plan, cross-sectional view, of a prior art electro-kinetic air transporter-conditioner system.

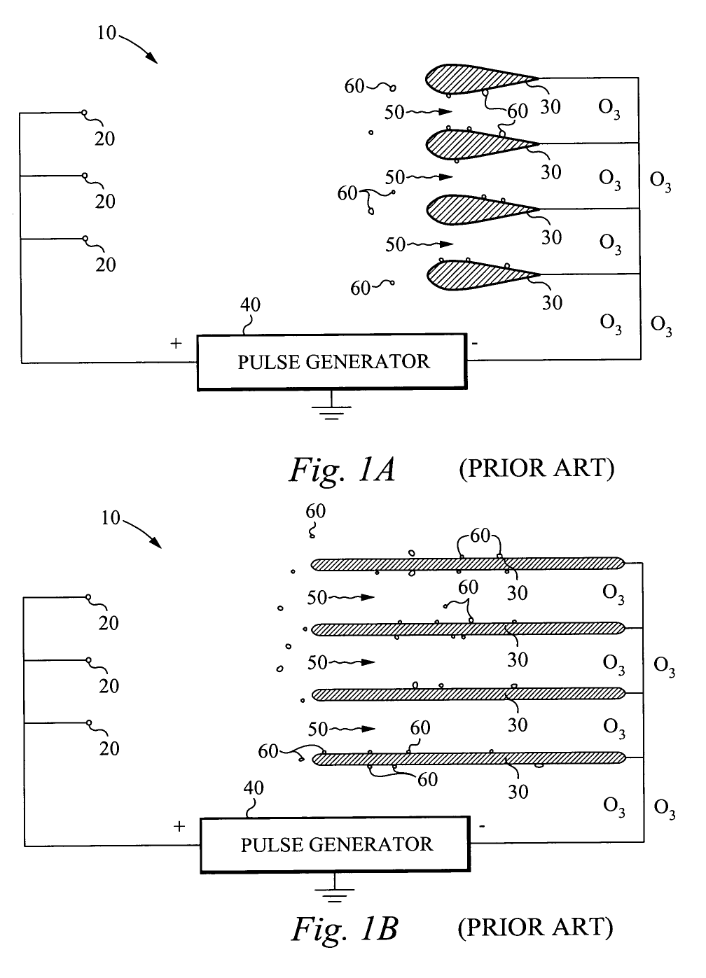

FIG. 1B illustrates a plan, cross-sectional view of a prior art electro-kinetic air transporter-conditioner system.

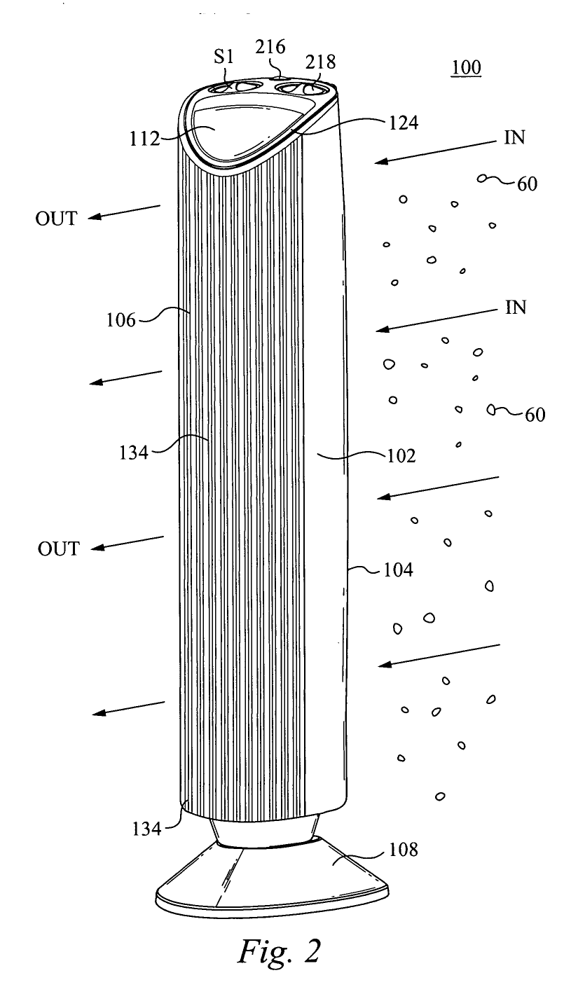

FIG. 2 illustrates a perspective view of the system in accordance with one embodiment of the present invention.

FIG. 3 illustrates a plan view of the electrode assembly in accordance with one embodiment of the present invention.

FIG. 4 illustrates an electrical block diagram of the high voltage power source of one embodiment of the present invention.

FIG. 5 illustrates an electrical block diagram of the high voltage power source in accordance with one embodiment of the present invention.

FIG. 6 illustrates an exploded view of the system shown in FIG. 2 in accordance with one embodiment of the present invention.

FIG. 7 illustrates a perspective view of the rear of the system with the germicidal lamp exposed in accordance with one embodiment of the present invention.

FIG. 8 illustrates a top view of the germicidal lamp in accordance with one embodiment of the present invention.

FIGS. 9-11 illustrate the system with the germicidal lamp positioned in various locations in accordance with one embodiment.

FIG. 12A-12B illustrate plan views of the germicidal lamp and engaging receptacle in accordance with one embodiment of the present invention.

FIG. 12C illustrates a perspective view of the engaging receptacle in accordance with one embodiment of the present invention.

FIG. 12D illustrates a perspective view of the germicidal lamp in accordance with one embodiment of the present invention.

FIG. 13 illustrates a perspective view of the front grill with trailing electrodes thereon in accordance with one embodiment of the present invention.

### DETAILED DESCRIPTION OF THE PRESENT INVENTION

An air transporting and/or conditioning system comprising a housing, an emitter electrode configured within the housing, a collector electrode configured within the housing and positioned downstream from the emitter electrode, and a integrally shielded germicidal lamp to selectively direct UV light emitted therefrom. The system preferably includes a driver electrode which is preferably removable from the housing through a side portion of the housing. Preferably, the driver electrode is insulated with a dielectric material and/or a catalyst. Preferably, a removable trailing electrode is configured within the housing and downstream of the collector electrode. Preferably, a first voltage source electrically is coupled to the emitter electrode and the collector electrode, and a second voltage source electrically is coupled to the trailing electrode. The second voltage source is independently and selectively controllable of the first voltage source.

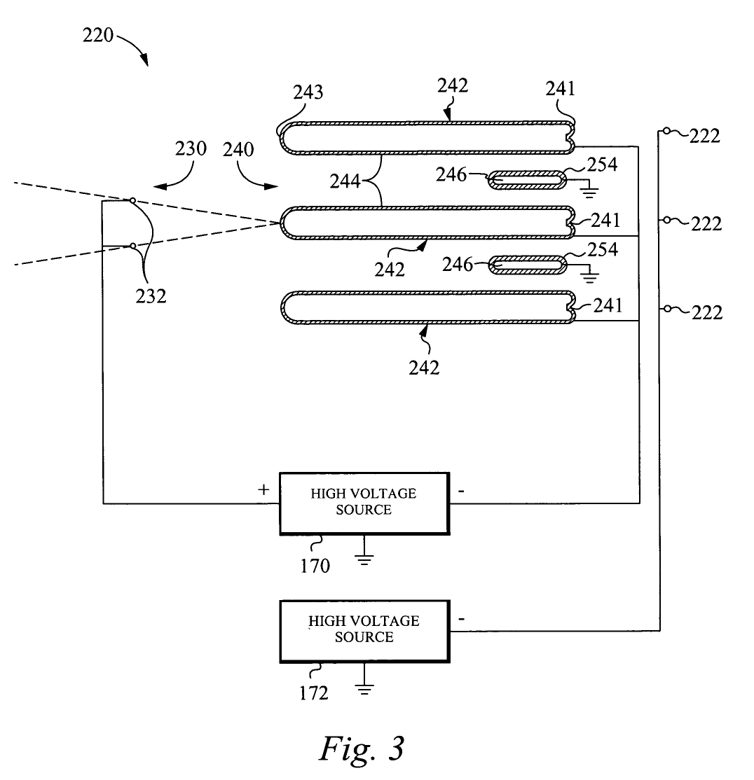

FIG. 2 depicts one embodiment of the air transporter-conditioner system 100 whose housing 102 preferably includes a removable rear-located intake grill 104, a removable front-located exhaust grill 106, and a base pedestal 108. Alternatively, a single grill provides both an air intake and an air exhaust with an air inlet channel and an air exhaust channel communicating with the grill and the air movement system within. The housing 102 is preferably freestanding and/or upstandingly vertical and/or elongated. The general shape of the housing 102 in the embodiment shown in FIG. 2 is that of an oval cross-section. Alternatively, the housing 102 includes a differently shaped cross-section such as, but not limited to, a rectangular shape, a figure-eight shape, an egg shape, a tear-drop shape, or circular shape.

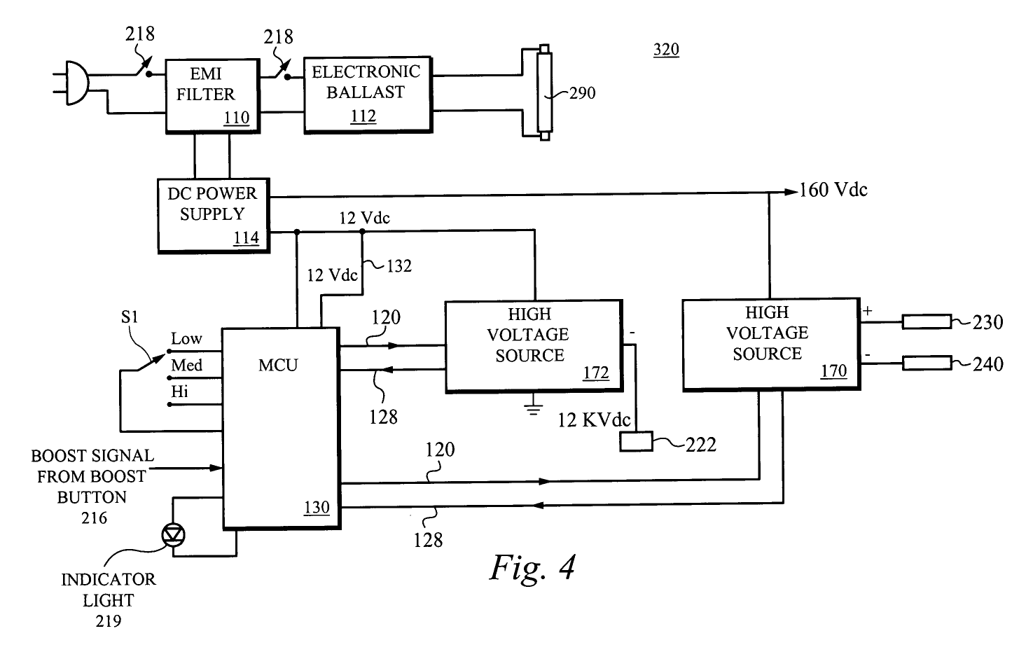

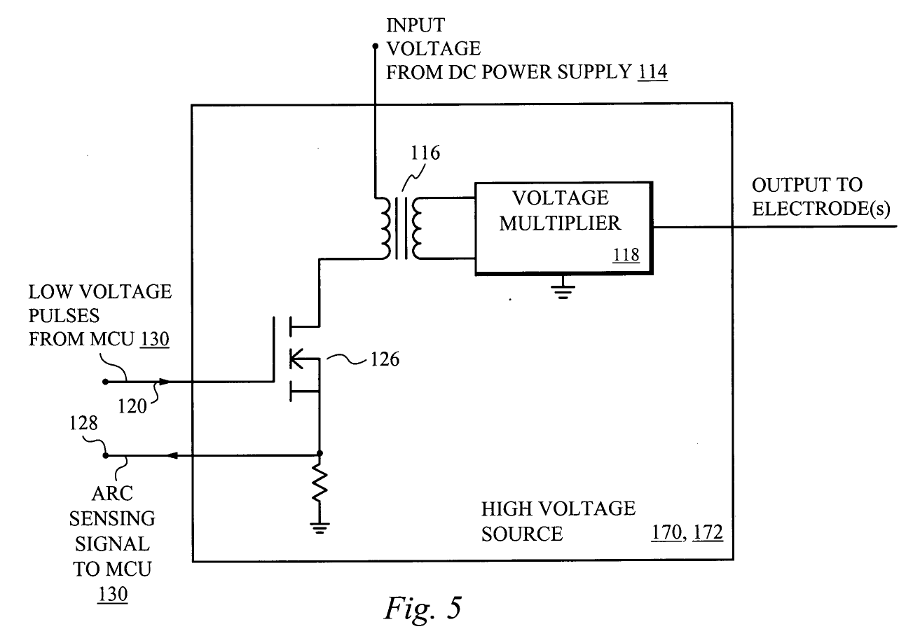

Internal to the transporter housing 102 is an air movement system which preferably includes an ion generating unit 220 (FIG. 3), also referred to as an electrode assembly. The ion generating unit 220 (FIG. 3) is self-contained in that, other than ambient air, nothing is required from beyond the housing 102, save external operating potential, for operation of the present invention. In one embodiment, the air movement system includes a fan utilized to supplement and/or replace the movement of air caused by the operation of the ion generator 220. The system 100 includes a germicidal lamp (FIG. 4) which reduces the amount of microorganisms exposed to the lamp when passed through the system 100. The germicidal lamp 290 (FIG. 4) is effective in diminishing or destroying bacteria, germs, and viruses to which it is exposed.

The ion generating unit 220 is preferably powered by an AC:DC power supply. The AC:DC power supply is energizable or excitable using a switch S1. S1 is conveniently located at the top 124 of the housing 102. The function dial 218 enables a user to operate the germicidal lamp 290 (FIG. 4). In particular, the user can select the dial 218 to “ON,” “ON/GP,” or “OFF.” In the “ON” setting, the germicidal lamp 290 does not operate or emit UV light, although the electrode assembly 220 operates. In the “ON/GP” setting, the germicidal lamp 290 operates to remove or kill bacteria within the airflow while the electrode assembly 220 operates. The electrode assembly 220 as well as the germicidal lamp 290 do not operate when the function dial 218 is set to the “OFF” setting. In addition, located preferably on top 124 of the housing 102 is a boost button 216 which can boost the ion output of the ion generator 220, as will be discussed below.

Both the inlet and the outlet grills 104, 106 are covered by fins 134, also referred to as louvers. In accordance with one embodiment, each fin 134 is a thin ridge spaced-apart from the next fin 134, so that each fin 134 creates minimal resistance as air flows through the housing 102. As shown in FIG. 2, the fins 134 are vertical and are directed along the elongated vertical upstanding housing 102 of the system 100, in one embodiment. Alternatively, the fins 134 are perpendicular to the elongated housing 102 and are configured horizontally. In one embodiment, the inlet and outlet fins 134 are aligned to give the unit a “see through” appearance while preventing an individual from viewing the UV light directly emitted from the germicidal lamp 290, as discussed below. Thus, a user can safely “see through” the system 100 from the inlet 104 to the outlet 106 or vice versa. The user will see no moving parts within the housing, but just a quiet unit that cleans the air passing therethrough.

There is preferably no distinction between grills 104 and 106, except their location relative to the collector electrodes 242 (FIG. 3). Alternatively, the grills 104 and 106 are configured differently and are distinct from one another. The grills 104, 106 serve to ensure that an adequate flow of ambient air is drawn into or made available to the system 100 and that an adequate flow of ionized air that includes appropriate amounts of ozone flows out from the system 100 via the exhaust grill 106. Thus, the IN flow preferably enters via grill(s) 104 and that the OUT flow exits via grill(s) 106 as shown in FIG. 2.

When the system 100 is energized by activating switch S1, high voltage or high potential output by the ion generator 220 produces at least ions within the system 100. The “IN” notation in FIG. 2 denotes the intake of ambient air with particulate matter 60 through the inlet grill 104. The “OUT” notation in FIG. 2 denotes the outflow of cleaned air through the exhaust grill 106 substantially devoid of the particulate matter 60.

FIG. 3 illustrates a plan view of the electrode assembly in accordance with one embodiment of the present invention. The electrode assembly 220 is shown to include the first electrode set 230, having the emitter electrodes 232, and the second electrode set 240, having the collector electrodes 242, preferably downstream from the first electrode set 230. In the embodiment shown in FIG. 3, the electrode assembly 220 also includes a set of driver electrodes 246 located interstitially between the collector electrodes 242. It is preferred that the electrode assembly 220 additionally includes a set of trailing electrodes 222 downstream from the collector electrodes 242. It is preferred that the number N1 of emitter electrodes 232 in the first set 230 differ by one relative to the number N2 of collector electrodes 242 in the second set 240. Preferably, the system 100 includes a greater number of collector electrodes 242 than emitter electrodes 232. However, if desired, additional emitter electrodes 232 are alternatively positioned at the outer ends of set 230 such that N1>N2, e.g., five emitter electrodes 232 compared to four collector electrodes 242. Alternatively, instead of multiple electrodes, single electrodes or single conductive surfaces are substituted. It is apparent that other numbers and arrangements of emitter electrodes 232, collector electrodes 242, trailing electrodes 222 and driver electrodes 246 are alternatively configured in the electrode assembly 220 in other embodiments.

The material(s) of the electrodes 232 and 242 should conduct electricity and be resistant to the corrosive effects from the application of high voltage, but yet be strong and durable enough to be cleaned periodically. In one embodiment, the emitter electrodes 232 are preferably fabricated from tungsten. Tungsten is sufficiently robust in order to withstand cleaning, has a high melting point to retard breakdown due to ionization, and has a rough exterior surface that promotes efficient ionization. The collector electrodes 242 preferably have a highly polished exterior surface to minimize unwanted point-to-point radiation. As such, the collector electrodes 242 are fabricated from stainless steel and/or brass, among other appropriate materials. The polished surface of electrodes 232 also promotes ease of electrode cleaning. The materials and construction of the electrodes 232 and 242, allow the electrodes 232, 242 to be light weight, easy to fabricate, and lend themselves to mass production. Further, electrodes 232 and 242 described herein promote more efficient generation of ionized air, and appropriate amounts of ozone.

As shown in FIG. 3, one embodiment of the present invention includes a first high voltage source (HVS) 170 and a second high power voltage source 172. The positive output terminal of the first HVS 170 is coupled to the emitter electrodes 232, and the negative output terminal of first HVS 170 is coupled to the collector electrodes 242. This coupling polarity has been found to work well and minimizes unwanted audible electrode vibration or hum. It is noted that in some embodiments, one port, such as the negative port, of the high voltage power supply can in fact be the ambient air. Thus, the electrodes 242 in the second set 240 need not be connected to the first HVS 170 using a wire. Nonetheless, there will be an “effective connection” between the collector electrodes 242 and one output port of the first HVS 170, in this instance, via ambient air. Alternatively, the negative output terminal of first HVS 170 is connected to the first electrode set 230 and the positive output terminal is connected to the second electrode set 240.

When voltage or pulses from the first HVS 170 are generated across the first and second electrode sets 230 and 240, a plasma-like field is created surrounding the electrodes 232 in first set 230. This electric field ionizes the ambient air between the first and the second electrode sets 230, 240 and establishes an “OUT” airflow that moves towards the second electrodes 240, which is herein referred to as the ionization region.

Ozone and ions are generated simultaneously by the first electrodes 232 as a function of the voltage potential from the HVS 170. Ozone generation is increased or decreased by respectively increasing or decreasing the voltage potential at the first electrode set 230. Coupling an opposite polarity voltage potential to the second electrodes 242 accelerates the motion of ions from the first set 230 to the second set 240, thereby producing the airflow in the ionization region. Molecules as well as particulates in the air thus become ionized with the charge emitted by the emitter electrodes 232 as they pass by the electrodes 232. As the ions and ionized particulates move toward the second set 240, the ions and ionized particles push or move air molecules toward the second set 240. The relative velocity of this motion is increased, by way of example, by increasing the voltage potential at the second set 240 relative to the potential at the first set 230. Therefore, the collector electrodes 242 collect the ionized particulates in the air, thereby allowing the system 100 to output cleaner, fresher air.

As shown in the embodiment in FIG. 3, at least one output trailing electrode 222 is electrically coupled to the second HVS 172. The trailing electrode 222 generates a substantial amount of negative ions, because the electrode 222 is coupled to relatively negative high potential. In one embodiment, the trailing electrode(s) 222 is a wire positioned downstream from the second electrodes 242. In one embodiment, the electrode 222 has a pointed shape in the side profile (e.g., a triangle) as described in U.S. patent application Ser. No. 10/074,347 which is incorporated by reference above.

The negative ions produced by the trailing electrode 222 neutralize excess positive ions otherwise present in the output airflow, such that the OUT flow has a net negative charge. The trailing electrodes 222 are preferably made of stainless steel, copper, or other conductor material. The inclusion of one electrode 222 has been found sufficient to provide a sufficient number of output negative ions. However, multiple trailing wire electrodes 222 are utilized in another embodiment. More details regarding the trailing electrode 222 are described in the 60/590,735 application, which is incorporated by reference above.

The use of the driver electrodes 246 increase the particle collection efficiency of the electrode assembly 220 and reduces the percentage of particles that are not collected by the collector electrode 242. This is due to the driver electrode 246 pushing particles in air flow toward the inside surface 244 of the adjacent collector electrode(s) 242, which is referred to herein as the collecting region. The driver electrode 246 is preferably insulated which further increases particle collection efficiency.

As stated above, the system of the present invention will also produce ozone (O₃). In accordance with one embodiment of the present invention, ozone production is reduced by preferably coating the internal surfaces of the housing with an ozone reducing catalyst. Exemplary ozone reducing catalysts include manganese dioxide and activated carbon. Commercially available ozone reducing catalysts such as PremAir™ manufactured by Englehard Corporation of Iselin, N.J., is alternatively used. Some ozone reducing catalysts are electrically conductive, while others are not electrically conductive (e.g., manganese dioxide). Preferably the ozone reducing catalysts should have a dielectric strength of at least 1000 V/mil (one-hundredth of an inch).

The insulated driver electrode 246 includes an electrically conductive electrode 253 that is coated with an insulating dielectric material 254. In embodiments where the driver electrode 246 is not insulated, the driver electrode 246 simply includes the electrically conductive electrode 253. In accordance with one embodiment of the present invention, the insulating dielectric material 254 is a heat shrink material (e.g. flexible polyolefin material). In another embodiment, the dielectric material 254 is an insulating varnish, lacquer or resin. Other possible dielectric materials 254 that can be used to insulate the driver electrode 253 include, but are not limited to, ceramic, porcelain enamel or fiberglass.

In one embodiment, the driver electrodes 246 are electrically connected to ground as shown in FIG. 3. Although the grounded drivers 246 do not receive a charge from either the first or second HVS 170, 172, the drivers 246 may still deflect positively charged particles toward the collector electrodes 242. In another embodiment, the driver electrodes 246 are positively charged. In yet another embodiment, the driver electrodes 246 are electrically coupled to the negative terminal of either the first or second HVS 170, 172, whereby the driver electrodes 246 are preferably charged at a voltage that is less than the negatively charged collector electrodes 242. More details regarding the insulated driver electrodes 246 are described in the 60/590,960 application, which is incorporated by reference above.

FIG. 4 illustrates an electrical circuit diagram for the system 100, according to one embodiment of the present invention. The system 100 has an electrical power cord that plugs into a common electrical wall socket that provides a nominal 110V AC. An electromagnetic interference (EMI) filter 110 is placed across the incoming nominal 110V AC line to reduce and/or eliminate high frequencies generated by the various circuits within the system 100, such as the electronic ballast 112. In one embodiment, the electronic ballast 112 is electrically connected to a germicidal lamp 290 (e.g. an ultraviolet lamp) to regulate, or control, the flow of current through the lamp 290. A switch 218 is used to turn the lamp 290 on or off. The EMI Filter 110 is well known in the art and does not require a further description. In another embodiment, the system 100 does not include the germicidal lamp 290, whereby the circuit diagram shown in FIG. 4 would not include the electronic ballast 112, the germicidal lamp 290, nor the switch 218 used to operate the germicidal lamp 290.

The EMI filter 110 is coupled to a DC power supply 114. The DC power supply 114 is coupled to the first HVS 170 as well as the second high voltage power source 172. The high voltage power source can also be referred to as a pulse generator. The DC power supply 114 is also coupled to the micro-controller unit (MCU) 130. The MCU 130 can be, for example, a Motorola 68HC908 series micro-controller, available from Motorola. Alternatively, any other type of MCU is contemplated. The MCU 130 can receive a signal from the switch S1 as well as a boost signal from the boost button 216. The MCU 130 also includes an indicator light 219 which specifies when the electrode assembly is ready to be cleaned.

The DC Power Supply 114 is designed to receive the incoming nominal 110V AC and to output a first DC voltage (e.g., 160VDC) to the first HVS 170. The DC Power Supply 114 voltage (e.g., 160VDC) is also stepped down to a second DC voltage (e.g., 12VDC) for powering the micro-controller unit (MCU) 130, the HVS 172, and other internal logic of the system 100. The voltage is stepped down through a resistor network, transformer or other component.

As shown in FIG. 4, the first HVS 170 is coupled to the first electrode set 230 and the second electrode set 240 to provide a potential difference between the electrode sets. In one embodiment, the first HVS 170 is electrically coupled to the driver electrode 246, as described above. In addition, the first HVS 170 is coupled to the MCU 130, whereby the MCU receives arc sensing signals 128 from the first HVS 170 and provides low voltage pulses 120 to the first HVS 170. Also shown in FIG. 4 is the second HVS 172 which provides a voltage to the trailing electrodes 222. In addition, the second HVS 172 is coupled to the MCU 130, whereby the MCU receives arc sensing signals 128 from the second HVS 172 and provides low voltage pulses 120 to the second HVS 172.

In accordance with one embodiment of the present invention, the MCU 130 monitors the stepped down voltage (e.g., about 12VDC), which is referred to as the AC voltage sense signal 132 in FIG. 4, to determine if the AC line voltage is above or below the nominal 110V AC, and to sense changes in the AC line voltage. For example, if a nominal 110V AC increases by 10% to 121V AC, then the stepped down DC voltage will also increase by 10%. The MCU 130 can sense this increase and then reduce the pulse width, duty cycle and/or frequency of the low voltage pulses to maintain the output power (provided to the HVS 170) to be the same as when the line voltage is at 110V AC. Conversely, when the line voltage drops, the MCU 130 can sense this decrease and appropriately increase the pulse width, duty cycle and/or frequency of the low voltage pulses to maintain a constant output power. Such voltage adjustment features of the present invention also enable the same system 100 to be used in different countries that have different nominal voltages than in the United States (e.g., in Japan the nominal AC voltage is 100V AC).

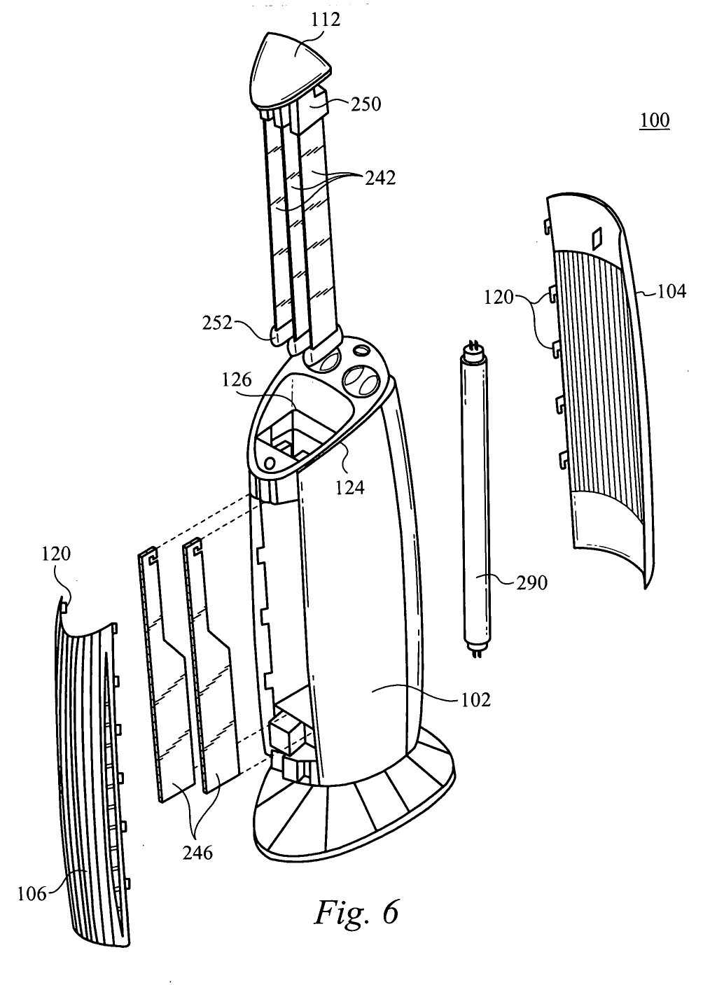

FIG. 5 illustrates a schematic block diagram of the high voltage power supply in accordance with one embodiment of the present invention. For the present description, the first and second HVSs 170, 172 include the same or similar components as that shown in FIG. 5. However, it is apparent to one skilled in the art that the first and second HVSs 170, 172 are alternatively comprised of different components from each other as well as those shown in FIG. 5. The various circuits and components comprising the first and second HVS 170, 172 can, for example, be fabricated on a printed circuit board mounted within housing 210. The MCU 130 can be located on the same circuit board or a different circuit board.

In the embodiment shown in FIG. 5, the HVSs 170, 172 include an electronic switch 126, a step-up transformer 116 and a voltage multiplier 118. The primary side of the step-up transformer 116 receives the DC voltage from the DC power supply 114. For the first HVS 170, the DC voltage received from the DC power supply 114 is approximately 160 Vdc. For the second HVS 172, the DC voltage received from the DC power supply 114 is approximately 12 Vdc. An electronic switch 126 receives low voltage pulses 120 (of perhaps 20-25 KHz frequency) from the MCU 130. Such a switch is shown as an insulated gate bipolar transistor (IGBT) 126. The IGBT 126, or other appropriate switch, couples the low voltage pulses 120 from the MCU 130 to the input winding of the step-up transformer 116. The secondary winding of the transformer 116 is coupled to the voltage multiplier 118, which outputs the high voltage pulses to the electrode(s). For the first HVS 170, the electrode(s) are the emitter and collector electrode sets 230 and 240. For the second HVS 172, the electrode(s) are the trailing electrodes 222. In general, the IGBT 126 operates as an electronic on/off switch. Such a transistor is well known in the art and does not require a further description.

When driven, the first and second HVSs 170, 172 receive the low input DC voltage from the DC power supply 114 and the low voltage pulses from the MCU 130 and generate high voltage pulses of preferably at least 5 KV peak-to-peak with a repetition rate of about 20 to 25 KHz. The voltage multiplier 118 in the first HVS 170 outputs between 5 to 9 KV to the first set of electrodes 230 and between −6 to −18 KV to the second set of electrodes 240. In the preferred embodiment, the emitter electrodes 232 receive approximately 5 to 6 KV whereas the collector electrodes 242 receive approximately −9 to −10 KV. The voltage multiplier 118 in the second HVS 172 outputs approximately −12 KV to the trailing electrodes 222. In one embodiment, the driver electrodes 246 are preferably connected to ground. It is within the scope of the present invention for the voltage multiplier 118 to produce greater or smaller voltages. The high voltage pulses preferably have a duty cycle of about 10%-15%, but may have other duty cycles, including a 100% duty cycle.

The MCU 130 is coupled to a control dial S1, as discussed above, which can be set to a LOW, MEDIUM or HIGH airflow setting as shown in FIG. 4. The MCU 130 controls the amplitude, pulse width, duty cycle and/or frequency of the low voltage pulse signal to control the airflow output of the system 100, based on the setting of the control dial S1. To increase the airflow output, the MCU 130 can be set to increase the amplitude, pulse width, frequency and/or duty cycle. Conversely, to decrease the airflow output rate, the MCU 130 is able to reduce the amplitude, pulse width, frequency and/or duty cycle. In accordance with one embodiment, the low voltage pulse signal 120 has a fixed pulse width, frequency and duty cycle for the LOW setting, another fixed pulse width, frequency and duty cycle for the MEDIUM setting, and a further fixed pulse width, frequency and duty cycle for the HIGH setting.

In accordance with one embodiment of the present invention, the low voltage pulse signal 120 modulates between a predetermined duration of a “high” airflow signal and a “low” airflow signal. It is preferred that the low voltage signal modulates between a predetermined amount of time when the airflow is to be at the greater “high” flow rate, followed by another predetermined amount of time in which the airflow is to be at the lesser “low” flow rate. This is preferably executed by adjusting the voltages provided by the first HVS to the first and second sets of electrodes for the greater flow rate period and the lesser flow rate period. This produces an acceptable airflow output while limiting the ozone production to acceptable levels, regardless of whether the control dial S1 is set to HIGH, MEDIUM or LOW. For example, the “high” airflow signal can have a pulse width of 5 microseconds and a period of 40 microseconds (i.e., a 12.5% duty cycle), and the “low” airflow signal can have a pulse width of 4 microseconds and a period of 40 microseconds (i.e., a 10% duty cycle).

In general, the voltage difference between the first set 230 and the second set 240 is proportional to the actual airflow output rate of the system 100. Thus, the greater voltage differential is created between the first and second set electrodes 230, 240 by the “high” airflow signal, whereas the lesser voltage differential is created between the first and second set electrodes 230, 240 by the “low” airflow signal. In one embodiment, the airflow signal causes the voltage multiplier 118 to provide between 5 and 9 KV to the first set electrodes 230 and between −9 and −10 KV to the second set electrodes 240. For example, the “high” airflow signal causes the voltage multiplier 118 to provide 5.9 KV to the first set electrodes 230 and −9.8 KV to the second set electrodes 240. In the example, the “low” airflow signal causes the voltage multiplier 118 to provide 5.3 KV to the first set electrodes 230 and −9.5 KV to the second set electrodes 240. It is within the scope of the present invention for the MCU 130 and the first HVS 170 to produce voltage potential differentials between the first and second sets electrodes 230 and 240 other than the values provided above and is in no way limited by the values specified.

In accordance with the preferred embodiment of the present invention, when the control dial S1 is set to HIGH, the electrical signal output from the MCU 130 will continuously drive the first HVS 170 and the airflow, whereby the electrical signal output modulates between the “high” and “low” airflow signals stated above (e.g. 2 seconds “high” and 10 seconds “low”). When the control dial S1 is set to MEDIUM, the electrical signal output from the MCU 130 will cyclically drive the first HVS 170 (i.e. airflow is “On”) for a predetermined amount of time (e.g., 20 seconds), and then drop to a zero or a lower voltage for a further predetermined amount of time (e.g., a further 20 seconds). It is to be noted that the cyclical drive when the airflow is “On” is preferably modulated between the “high” and “low” airflow signals (e.g. 2 seconds “high” and 10 seconds “low”), as stated above. When the control dial S1 is set to LOW, the signal from the MCU 130 will cyclically drive the first HVS 170 (i.e. airflow is “On”) for a predetermined amount of time (e.g., 20 seconds), and then drop to a zero or a lower voltage for a longer time period (e.g., 80 seconds). Again, it is to be noted that the cyclical drive when the airflow is “On” is preferably modulated between the “high” and “low” airflow signals (e.g. 2 seconds “high” and 10 seconds “low”), as stated above. It is within the scope and spirit of the present invention the HIGH, MEDIUM, and LOW settings will drive the first HVS 170 for longer or shorter periods of time. It is also contemplated that the cyclic drive between “high” and “low” airflow signals are durations and voltages other than that described herein.

Cyclically driving airflow through the system 100 for a period of time, followed by little or no airflow for another period of time (i.e. MEDIUM and LOW settings) allows the overall airflow rate through the system 100 to be slower than when the dial S1 is set to HIGH. In addition, cyclical driving reduces the amount of ozone emitted by the system since little or no ions are produced during the period in which lesser or no airflow is being output by the system. Further, the duration in which little or no airflow is driven through the system 100 provides the air already inside the system a longer dwell time, thereby increasing particle collection efficiency. In one embodiment, the long dwell time allows air to be exposed to a germicidal lamp, if present.

Regarding the second HVS 172, approximately 12 volts DC is applied to the second HVS 172 from the DC Power Supply 114. The second HVS 172 provides a negative charge (e.g. −12 KV) to one or more trailing electrodes 222 in one embodiment. However, it is contemplated that the second HVS 172 provides a voltage in the range of, and including, −10 KV to −60 KV in other embodiments. In one embodiment, other voltages produced by the second HVS 172 are contemplated.

In one embodiment, the second HVS 172 is controllable independently from the first HVS 170 (as for example by the boost button 216) to allow the user to variably increase or decrease the amount of negative ions output by the trailing electrodes 222 without correspondingly increasing or decreasing the amount of voltage provided to the first and second set of electrodes 230, 240. The second HVS 172 thus provides freedom to operate the trailing electrodes 222 independently of the remainder of the electrode assembly 220 to reduce static electricity, eliminate odors and the like. In addition, the second HVS 172 allows the trailing electrodes 222 to operate at a different duty cycle, amplitude, pulse width, and/or frequency than the electrode sets 230 and 240. In one embodiment, the user is able to vary the voltage supplied by the second HVS 172 to the trailing electrodes 222 at any time by depressing the button 216. In one embodiment, the user is able to turn on or turn off the second HVS 172, and thus the trailing electrodes 222, without affecting operation of the electrode assembly 220 and/or the germicidal lamp 290. It should be noted that the second HVS 172 can also be used to control electrical components other than the trailing electrodes 222 (e.g. driver electrodes and germicidal lamp).

As mentioned above, the system 100 includes a boost button 216. In one embodiment, the trailing electrodes 222 as well as the electrode sets 230, 240 are controlled by the boost signal from the boost button 216 input into the MCU 130. In one embodiment, as mentioned above, the boost button 216 cycles through a set of operating settings upon the boost button 216 being depressed. In the example embodiment discussed below, the system 100 includes three operating settings. However, any number of operating settings are contemplated within the scope of the invention.

The following discussion presents methods of operation of the boost button 216 which are variations of the methods discussed above. In particular, the system 100 will operate in a first boost setting when the boost button 216 is pressed once. In the first boost setting, the MCU 130 drives the first HVS 170 as if the control dial S1 was set to the HIGH setting for a predetermined amount of time (e.g., 6 minutes), even if the control dial S1 is set to LOW or MEDIUM (in effect overriding the setting specified by the dial S1). The predetermined time period may be longer or shorter than 6 minutes. For example, the predetermined period can also preferably be 20 minutes if a higher cleaning setting for a longer period of time is desired. This will cause the system 100 to run at a maximum airflow rate for the predetermined boost time period. In one embodiment, the low voltage signal modulates between the “high” airflow signal and the “low” airflow signal for predetermined amount of times and voltages, as stated above, when operating in the first boost setting. In another embodiment, the low voltage signal does not modulate between the “high” and “low” airflow signals.

In the first boost setting, the MCU 130 will also operate the second HVS 172 to operate the trailing electrode 222 to generate ions, preferably negative, into the airflow. In one embodiment, the trailing electrode 222 will preferably repeatedly emit ions for one second and then terminate for five seconds for the entire predetermined boost time period. The increased amounts of ozone from the boost level will further reduce odors in the entering airflow as well as increase the particle capture rate of the system 100. At the end of the predetermined boost period, the system 100 will return to the airflow rate previously selected by the control dial S1. It should be noted that the on/off cycle at which the trailing electrodes 222 operate are not limited to the cycles and periods described above.

In the example, once the boost button 216 is pressed again, the system 100 operates in the second setting, which is an increased ion generation or “feel good” mode. In the second setting, the MCU 130 drives the first HVS 170 as if the control dial S1 was set to the LOW setting, even if the control dial S1 is set to HIGH or MEDIUM (in effect overriding the setting specified by the dial S1). Thus, the airflow is not continuous, but “On” and then at a lesser or zero airflow for a predetermined amount of time (e.g. 6 minutes). In addition, the MCU 130 will operate the second HVS 172 to operate the trailing electrode 222 to generate negative ions into the airflow. In one embodiment, the trailing electrode 222 will repeatedly emit ions for one second and then terminate for five seconds for the predetermined amount of time. It should be noted that the on/off cycle at which the trailing electrodes 222 operate are not limited to the cycles and periods described above.

In the example, upon the boost button 216 being pressed again, the MCU 130 will operate the system 100 in a third operating setting, which is a normal operating mode. In the third setting, the MCU 130 drives the first HVS 170 depending on the which setting the control dial S1 is set to (e.g. HIGH, MEDIUM or LOW). In addition, the MCU 130 will operate the second HVS 172 to operate the trailing electrode 222 to generate ions, preferably negative, into the airflow at a predetermined interval. In one embodiment, the trailing electrode 222 will repeatedly emit ions for one second and then terminate for nine seconds. In another embodiment, the trailing electrode 222 does not operate at all in this mode. The system 100 will continue to operate in the third setting by default until the boost button 216 is pressed. It should be noted that the on/off cycle at which the trailing electrodes 222 operate are not limited to the cycles and periods described above.

In one embodiment, the present system 100 operates in an automatic boost mode upon the system 100 being initially plugged into the wall and/or initially being turned on after being off for a predetermined amount of time. In particular, upon the system 100 being turned on, the MCU 130 automatically drives the first HVS 170 as if the control dial S1 was set to the HIGH setting for a predetermined amount of time, as discussed above, even if the control dial S1 is set to LOW or MEDIUM, thereby causing the system 100 to run at a maximum airflow rate for the amount of time. In addition, the MCU 130 automatically operates the second HVS 172 to operate the trailing electrode 222 at a maximum ion emitting rate to generate ions, preferably negative, into the airflow for the same amount of time. This configuration allows the system 100 to effectively clean stale, pungent, and/or polluted air in a room which the system 100 has not been continuously operating in. This feature improves the air quality at a faster rate while emitting negative “feel good” ions to quickly eliminate any odor in the room. Once the system 100 has been operating in the first setting boost mode, the system 100 automatically adjusts the airflow rate and ion emitting rate to the third setting (i.e. normal operating mode). For example, in this initial plug-in or initial turn-on mode, the system can operate in the high setting for 20 minutes to enhance the removal of particulates and to more rapidly clean the air as well as deodorize the room.

In addition, the system 100 will include an indicator light which informs the user what mode the system 100 is operating in when the boost button 216 is depressed. In one embodiment, the indicator light is the same as the cleaning indicator light 219 discussed above. In another embodiment, the indicator light is a separate light from the indicator light 219. For example only, the indicator light will emit a blue light when the system 100 operates in the first setting. In addition, the indicator light will emit a green light when the system 100 operates in the second setting. In the example, the indicator light will not emit a light when the system 100 is operating in the third setting.

The MCU 130 provides various timing and maintenance features in one embodiment. For example, the MCU 130 can provide a cleaning reminder feature (e.g., a 2 week timing feature) that provides a reminder to clean the system 100 (e.g., by causing indicator light 219 to turn on amber, and/or by triggering an audible alarm that produces a buzzing or beeping noise). The MCU 130 can also provide arc sensing, suppression and indicator features, as well as the ability to shut down the first HVS 170 in the case of continued arcing. Details regarding arc sensing, suppression and indicator features are described in U.S. patent application Ser. No. 10/625,401 which is incorporated by reference above.

In addition, the MCU 130 includes a lamp timing feature which notifies the user that the lamp 290 is in need of replacement. In particular, upon the timing feature counting a predetermined duration (e.g. 8000 operating hours), the MCU 130 will notify the user that the lamp 290 should be replaced. It is preferred that the timing feature of the MCU 130 tolls the counting while the unit is off or unplugged. In one embodiment, the MCU 130 notifies the user using the indicator light 219 discussed above, whereby the indicator light turns a different color and/or begins flashing. In another embodiment, the system 100 includes a separate indicator. The lamp timing feature of the MCU 130 is preferably set by the manufacturer to the normal operating life of the lamp 290.

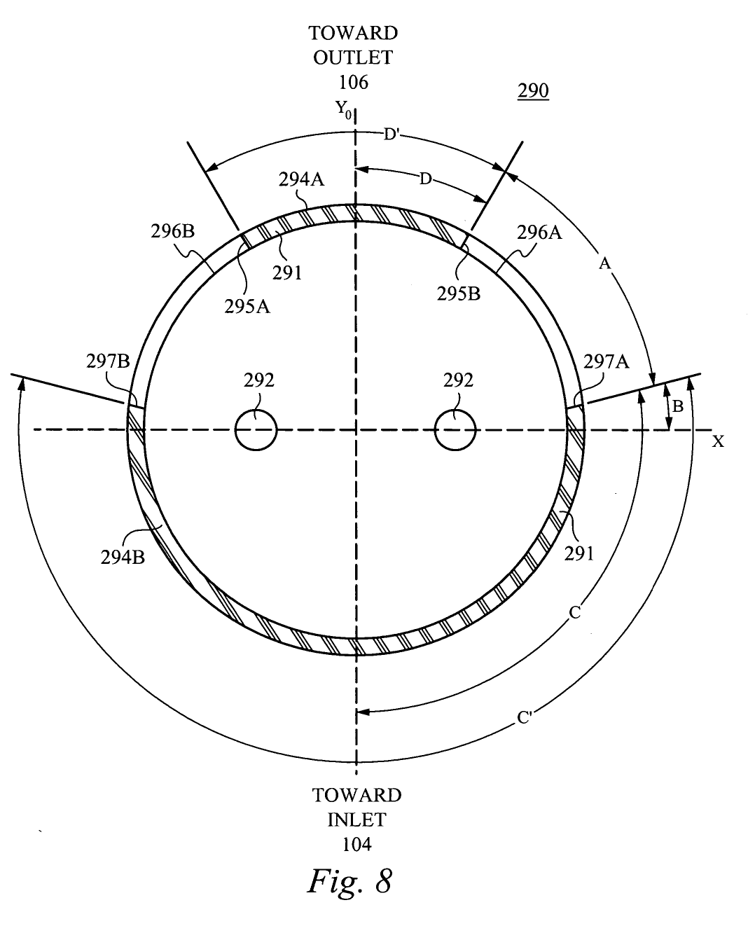

The timing feature of the MCU 130 is preferably reset by the user. In one embodiment, the timing feature is reset by performing a combination of steps. This prevents the user from inadvertently resetting the timer. For example only, the timing feature is able to be reset by simultaneously pressing the boost button 216 and turning the S1 switch to HIGH while the unit is off. The “high” airflow signal and the boost button signal enter the MCU 130 to thereby reset the timer circuit. In another embodiment, the timer feature is reset by a mechanical switch in the receptacle 300 (FIG. 7), whereby simply removing and/or inserting the lamp 290 into the receptacle 300 resets the timer circuit.

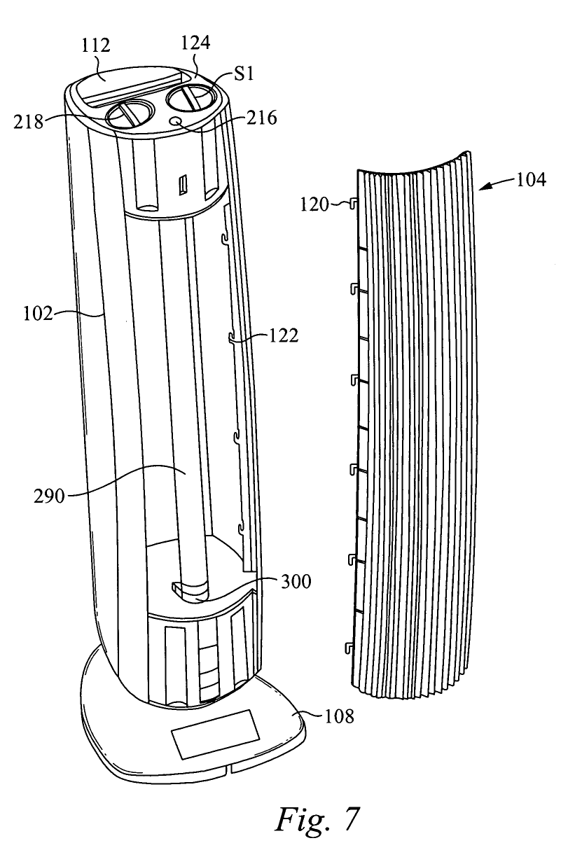

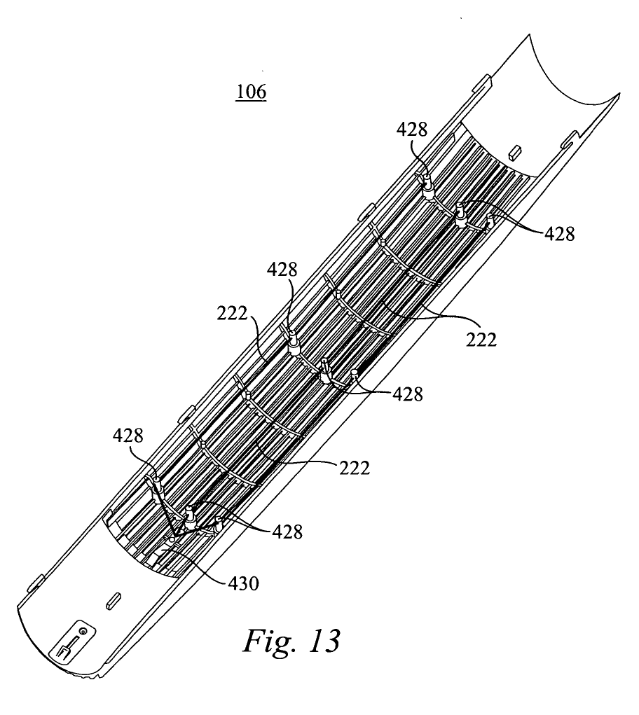

FIG. 6 illustrates an exploded view of the system 100 in accordance with one embodiment of the present invention. In particular, FIG. 6 illustrates the housing 102, the rear intake grill 104 (also referred to as inlet), the front exhaust grill 106 (also referred to outlet), the collector electrodes 242, the driver electrodes 246 and the germicidal lamp 290. The system 100 also includes one or more trailing electrodes 222 (FIG. 13). As shown in the embodiment in FIG. 6, the upper surface of housing 102 includes a user-liftable handle member 112 to lift the collector electrodes 242 from the housing 102. In the embodiment shown in FIG. 6, the lifting member 112 lifts the collector electrodes 242 upward, thereby causing the collector electrodes 242 to telescope out of the aperture 126 in the top surface 124 of the housing 102 and, and if desired, out of the system 100 for cleaning. In addition, the driver electrodes 246 are removable from the housing 102 horizontally, as shown in FIG. 6, when the exhaust grill 106 is removed from the housing 102. Alternatively or additionally, the driver electrodes are removable vertically from the housing 102 as further discussed in U.S. patent application Ser. No. 60/590,688, which is incorporated by reference above.

The housing 102 is preferably made from a lightweight inexpensive material, ABS plastic for example. Considering that a germicidal lamp 290 is located within the housing 102, the material must be able to withstand prolonged exposure to class UV-C light. Non-“hardened” material will degenerate over time if exposed to light such as UV-C. By way of example only, the housing 102 may be manufactured from CYCLOLAC7 ABS Resin (material designation VW300(f2)), which is manufactured by General Electric Plastics Global Products, and is certified by UL Inc. for use with ultraviolet light. It is within the scope of the present invention to manufacture the housing 102 from other UV appropriate materials.

FIG. 7 illustrates a rear perspective view of the system 100 with the intake grill 104 removed from the housing 102. In one embodiment, the removable intake grill 104 allows a user to easily remove and replace the germicidal lamp 290 from the receptacle 300 in the housing 102 when the lamp 290 expires. In the embodiment in which the grill 104 is removable, the grill 104 has locking tabs 120 located on each side, along the entire length of the grill 104. The locking tabs 120, as shown in FIG. 7, are “L”-shaped. Each tab 120 extends away from the grill 104, inward towards the housing 102, and then projects downward, parallel with the edge of the grill 104. It is also within the spirit and scope of the invention to have differently-shaped tabs 120. Each tab 120 individually and slidably interlocks with recesses 122 formed within the housing 102. The grill 104 is preferably slid vertically upward until the tabs 120 disengage the recesses 122. The grill 104 is then pulled away from the housing 102 in a lateral direction, as shown in FIG. 7. Removing the grill 104 exposes the lamp 290 within the housing 102. In one embodiment, the grill 104 includes a safety mechanism, such as a rear proj ecting tab removed from a receiving slot, to shut the system 100 off when the grill 104 is removed.

In another embodiment, the germicidal lamp 290 is removable from the housing 102 by vertically lifting the germicidal lamp 290 out through the top surface 124. The lamp 290 is mounted to a lamp fixture that has circuit contacts which engage the circuit 320 (FIG. 4), such that the lamp 290 will shut the entire system 100 off when lifted out of the housing. In similar, but less convenient fashion, the lamp 290 maybe designed to be removed from the bottom of the housing 102. More details regarding removing the lamp 290 telescopically from the housing 102 are discussed in U.S. patent application Ser. No. 10/074,347 which is incorporated by reference above.

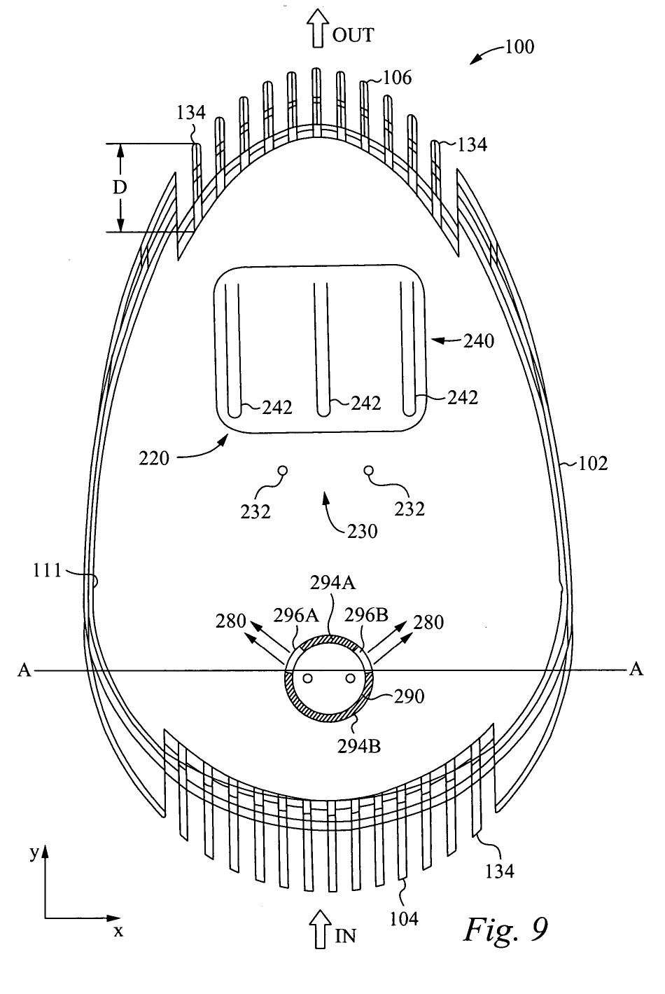

FIG. 8 illustrates a plan view of the preferred germicidal lamp 290 in accordance with one embodiment of the present invention. As shown in FIG. 8, the ends of the lamp 290 preferably include two lamp pins 292 which electrically connect the lamp 290 to the electronic ballast (FIG. 5). However, as discussed below, one or more ends of the lamp 290 may alternatively have additional pins.

The germicidal lamp 290 is preferably a UV-C lamp that preferably emits viewable light and radiation (in combination referred to as radiation or light 280) having wavelength of about 254 nm. This wavelength is effective in diminishing or destroying bacteria, germs, and viruses to which it is exposed. As shown in FIG. 8, the lamp 290 includes a shield 294 integrally configured which selectively directs UV light and radiation emitted by the lamp 290. Lamps 290 are commercially available. For example, the lamp 290 may be a 15 W tubular lamp measuring about 25 mm in diameter by about 43 cm in length. Other lamps that emit the desired wavelength are alternatively used. It should be noted that although the integrally shielded lamp shown and described herein is applied within an air conditioner system, the integrally shielded lamp 290 can be applied in any other application, system, or device within the scope of the present invention.

The lamp 290 shown in FIG. 8 includes two distinct shielded regions 294 as well as two distinct non-shielded regions 296. Any number of shielded or non-shielded regions, including only one, are alternatively contemplated. The shielded regions 294 of the lamp 290 are preferably coated with a shielding material 291 which prevents UV light and radiation emitted by the lamp 290 from passing therethrough. In one embodiment, the shielding material 291 is a coating which is disposed on the inner and/or outer surface of the germicidal lamp 290. In another embodiment, the shielding material 291 is formed in the glass housing of the lamp 290 during manufacturing of the lamp 290. In one embodiment, the shielding material 219 is formed between the inner and outer surfaces of the lamp 290. In another embodiment, the shielding material 219 is formed between and including the inner and outer surfaces of the lamp 290. The shielding material 291 of the lamp 290 is preferably made of titanium dioxide. However, it is within the scope of the present invention that the shielding material 291 be any appropriate material which blocks emission of UV light and radiation from the lamp 290. In one embodiment, the interior of the lamp 290 is lined with a reflective material in the areas where the shielding material 291 is disposed to increase the UV intensity through the non-shielded regions 296. In another embodiment, the interior of the lamp 290 is not lined with a reflective material. The shielding material 294 is disposed on the lamp 290 by known methods which are not discussed in detail herein.

As shown in the Figures, the shielding material 291 is disposed on predetermined locations of the lamp 290 such that the shielded regions 294 face the inlet and outlets 104, 106 and the non-shielded regions 296 face the inner walls 101 of the housing 102. The shielded locations 294 where the shielding material 291 is located depend on the location as well as the orientation of the lamp 290 within the housing 102 as discussed in more detail below. It is preferred that the shielded regions 294 cover the lamp 290 from the lamp's top end to the lamp's bottom end. Alternatively, the shielded regions 294 are not continuous from the top end to the bottom end of the lamp 290.

As stated above, the non-shielded regions 296 of the lamp 290 allow UV light and radiation to pass through. It is preferred that the lamp 290 is configured and oriented such that non-shielded regions 296 allow UV light and radiation to be emitted onto the inner surface 111 of the housing 102 away from the view of the user. Thus, the non-shielded regions 296 do not allow UV light and radiation to pass directly onto the inlet and outlet 104, 106 of the housing 102. The lamp 290 is thus oriented such that the shielded regions 294 face the inlet 104 and outlet 106, thereby preventing UV light and radiation from being directly emitted toward the inlet 104 and/or outlet 106 in which a user would be able to view the directly emitted light. In addition, the configuration of the louvers 134 as well as placement of the shielded regions 294 prevent an individual looking into the inlet 104 and/or outlet 106 from directly viewing the UV light and radiation emitted directly by the lamp 290. The integrally shielded lamp 290 of the present invention thus eliminates the need for light deflecting baffles or other housings which can simplify manufacturing of the system 100. Without such baffles and other housing shields, there is less structure in the housing that can potentially impede the flow of air from the inlet 104 to the outlet 106. In addition, the use of an integrally shielded lamp can provide the ability to specifically direct light to a desired location in the housing (e.g. collector electrodes) if desired.

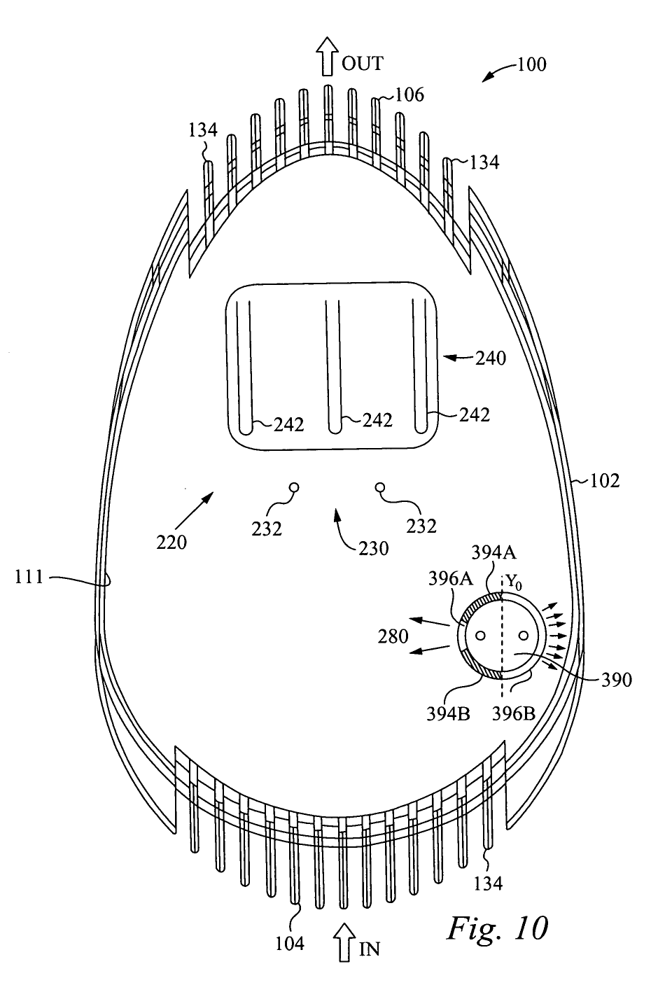

As shown in FIG. 9, the system includes the ion generator 220 along with the germicidal lamp 290 of FIG. 8 positioned upstream of the ion generator 220. In particular, the electrode assembly 220 is positioned near the outlet grill 106, whereas the germicidal lamp 290 is positioned near the inlet grill 104, preferably along line A-A. The germicidal lamp 290 is also shown placed directly in-line with both the inlet 104 and outlet 106. The housing 102 of the present system 100 is preferably designed to optimize the reduction of microorganisms within the airflow, whereby the efficacy of radiation 280 upon microorganisms depends upon the length of time such organisms are subjected to the radiation 280. Thus, the lamp 290 is preferably located within the housing 102 where the airflow is the slowest which is along line A-A. Line A-A designates the largest width and cross-sectional area of the housing 102, which is perpendicular to the airflow. By positioning the lamp 290 substantially along line A-A, the air will have the longest dwell time as it passes through the radiation 280 emitted by the lamp 290. It is, however, within the scope of the present invention to locate the lamp 290 anywhere within the housing 102, preferably upstream of the electrode assembly 220

It is desired to provide the inner surface of the housing 102 with an electrostatic shield to reduce detectable electromagnetic radiation. In one embodiment, a metal shield or metallic paint is preferably disposed within the housing 102, or regions of the interior of the housing 102. In one embodiment, the inner surface 111 has a non-smooth finish or a non-light reflecting finish or color. In general, when the UV rays emitted by the lamp 290 strikes the interior surface 111 of the housing 102, the radiation 280 is shifted from its emitted UV spectrum to an appropriate viewable spectrum. Thus, the potentially undesired UV portion of the light and radiation 280 which strikes the interior surface 111 will be absorbed by the surface 111, whereas the harmless UV portion of the radiation 280 will be disbursed as viewable light.

As discussed above in one example, the louvers 134 covering the inlet 104 and the outlet 106 also limit any angle of sight for the individual looking into the housing 102. The depth D of each fin 134 is preferably sufficient to prevent an individual from directly viewing the interior wall 111 when looking into the inlet and/or outlet grill 104, 106. Instead, the user will be to “see through” the device upon looking through the inlet and the outlet. It is to be understood that it is acceptable to see light or a glow coming from within housing 102 if the wavelength of the light renders it acceptable for viewing. Therefore, the configuration of the fins 134 as well as the lamp 290 allow an individual to look into the inlet 104 or the outlet 106 and be able to see light or glow which is not harmful to the individual.

Referring back to FIG. 8, specific areas of the lamp 290 are configured to include the shielding material 291 such that UV light is directed toward the inner surface 111 and away from the inlet 104 and the outlet 106. The particular lamp 290 in FIG. 8 is shown placed in the housing 102 in FIG. 9. The specific angles, arc lengths, and locations of the shielded regions 294 as well as the non-shielded regions 296 of the particular lamp 290 are discussed in relation to the Y₀ axis. The shielded and non-shielded regions of the lamp 290 shown for the embodiment in FIGS. 8 and 9 are preferably symmetrical about the Y axis. The lamp 290 has a front shielded region 294A which faces the outlet 106 when positioned in the housing as well as a rear shielded region 294B which faces the inlet 104 of the housing, as shown in FIGS. 8 and 9. A portion of the front shielded region 294A preferably has an arc-length of about 30 degrees clockwise from the Y₀ axis, shown as angle D, whereby Y₀ is the reference point of the angles discussed herein. As shown in FIG. 8, the remaining portion of the front shielded region 294A has an arc length of 30 degrees counterclockwise from the Y₀ axis (i.e. 330 degrees clockwise with respect to Y₀). Thus, for the embodiment of the lamp 290 shown in FIG. 8, the front shielded region 294A extends 60 degrees (shown as angle D′) from the left end 295A to the right end 295B, whereby the left end 295A is approximately at 330 degrees from the Y₀ axis and the right end 295B is approximately at 30 degrees from the Y₀ axis. It should be noted that the angles and arc-lengths discussed above are for one embodiment and are not to be construed to be limited thereto.

The rear shielded region 294B is shown in FIG. 8 extending between a right end 297A and a left end 297B which preferably faces the inlet of the housing. As shown in FIG. 8, the right end 297A of the rear shielded portion 291 is located approximately at 80 degrees from the Y₀ axis (angle B is preferably 10 degrees). Additionally, the left end 297B of the rear shielded portion 291 is approximately located at 280 degrees from the Y₀ axis. Thus, the rear shielded region 294B of the embodiment shown in FIG. 8 preferably has an arc-length of about 100 degrees (angle C) and an overall arc-length of approximately 200 degrees (angle C′). It should be noted that the angles and arc-lengths discussed above are for one embodiment and are not to be construed to be limited thereto.

The right non-shielded region 296A of the lamp 290 is located adjacent to the front and rear shielded regions and preferably has an arc-length of about 50 degrees with respect to the center of the lamp 290, which is shown as angle A. Thus, as shown in FIG. 8, the right non-shelded region 296A extends between the right end 295B of the front shielded region and the right end 297A of the rear shielded region 294B. Considering that the lamp 290 is symmetrical about the Y-axis, the lamp 290 also includes a left non-shielded region 296B has an arc-length of about 50 degrees with respect to the center. The non-shielded region 296A is located between the left end 295A of the front shielded region 294A and the left end 297B of the rear shielded region 294B in the embodiment shown in FIG. 8. As shown in FIG. 8, the right non-shielded region 296A has boundaries approximately at 30 degrees clockwise from the Y axis (adjacent to front shielded region 294A) and 80 degrees (adjacent to rear shielded region 294B) clockwise from the Y axis. As stated above, the lamp 290 in FIG. 8 is symmetrical about the Y axis. Therefore, the boundaries of the left non-shielded region 296B is located at approximately 30 degrees counter clockwise from the Y axis (adjacent to the rear shielded region 294B) and 80 degrees (adjacent to the front shielded region 294A) counter clockwise with respect to the Y axis. As stated above, it should be noted that the angles, locations and numbers of shielded and non-shielded regions discussed in relation to FIG. 8 are examples and are not meant to be limiting. It should also be noted that any other angles, locations and numbers of the shielded and non-shielded regions are contemplated.

The particular angles and locations of the shielded regions 294 as well as the non-shielded regions controls the location and amount of UV light and radiation 280 disbursed by the lamp 290 within the housing 102. In particular, the front shielded region 294A is located to face the outlet grill 106, whereby the angle of the front shielded region 294A (i.e. angle D) radially covers the lamp 290 to prevent undesirable UV light from being dispersed directly at the outlet grill 106. In addition, the rear shielded region 294B is located to face the inlet grill 104, whereby the angle of the rear shielded region 294B (i.e. angle C) radially covers the lamp 290 to prevent undesirable UV light to be dispersed directly at the inlet grill 104. The non-shielded regions 296A and 296B are oriented to face the inner walls 111 of the housing and away from the inlet and outlet grill 104, 106 such that an individual looking into the system 100 through the inlet 104 or outlet 106 would not be able to view UV light directly emitted by the lamp 290. The angles of the non-shielded regions 296 (i.e. angle A) are such that sufficient UV light is able to be emitted out of the lamp 290 to adequately neutralize microorganisms in the airflow.

In the embodiment shown in FIG. 10, the lamp 390 is located along the side of the housing 102. As the air enters the housing 102, the air is immediately exposed to the light 280 emitted by the lamp 390. In FIG. 10, the lamp 390 is configured and oriented such that the shielded regions 394A, 394B block UV light 280 from being directed toward the inlet 104 and outlet 106. The shape and depth D of the louvers 134 prevent an individual from seeing the lamp at an angle into the housing 102. Thus, the top shielded region 394A covers the portion of the lamp 390 which is viewable by an individual looking into the housing through the space between the louvers 134 in the outlet 106. Similarly, the rear shielded region 394B shields light emitted from the lamp 390 from being emitted or viewed through the space between the louvers 134 in the inlet 104.

Additionally, the non-shielded regions 396 of the lamp 390 are located to face the interior walls 111 of the housing 102. In particular, the non-shielded region 396A (about 50 degrees arc length) is oriented and has an appropriate radial width to direct light toward the inner wall 111 on the left side of the housing 102 without allowing undesired UV light from the lamp 290 to be viewed by an individual looking into the housing 102. Similarly, the non-shielded region 396B (about 160 degree in arc-length) is oriented and has an appropriate radial width to direct light toward the inner wall 111 on the right side of the housing 102. As shown in FIG. 10, a substantial portion 396B of the lamp 390 is out of the direct line of sight through the inlet 104 and the outlet 106, and the portion 396B is located near the right side of the housing 101. The portion 396B is thus not shielded, since almost all the light and radiation 280 emitted through the non-shielded portion 396B is immediately directed onto the inner wall 111 on the right side of the housing 102. In one embodiment, one non-shielded region 296 of the lamp 290 faces several light guides which further prevent the light 280 from shining directly towards the inlet 104 and the outlet 106 and also guide the light toward the opposing wall 111. More details of the light guides are described in the U.S. application Ser. No. 10/074,347 which is incorporated by reference above. It should be noted that the angles, locations and numbers of shielded and non-shielded regions discussed in relation to FIG. 10 are examples and are not meant to be limiting. It should also be noted that any other angles, locations and numbers of the shielded and non-shielded regions are contemplated.

As shown in FIG. 11, the inlet grill 104 includes multiple vertical slots 136 located along each side of a rear wall 138, whereby the slots 136 face in a direction perpendicular to the louvers 134 of the exhaust grill 106 and the general direction of the airflow through the system 100. Thus, air outside of the housing 102 travels in toward the inlet grill 104 and then enters the housing 102 in a perpendicular direction. The rear wall 138 is preferably a solid, opaque structure which does not allow light to pass through it. In one embodiment, the rear wall 138 of the inlet grill 104 is coated with the same material as the rest of the interior 111 of the housing to absorb and/or disburse the UV light emitted by the lamp 490. The lamp 490 in the embodiment in FIG. 11 has only one shielded region 494 which covers a substantial portion of the radial surface of the lamp 490 which faces the exhaust grill 106. In one embodiment, the shielded region 494 has an arc-length of about 70 degrees with respect to the center as with the lamp 290 discussed in FIG. 8. Since the rear wall 138 does not allow light to pass through and has the inlets 136 facing perpendicular to the outlet 106 and toward the inner walls 111 of the housing, an individual cannot see the non-shielded region of the lamp 490 by looking into the housing 102 through the inlet slots 136. Thus, the side of the lamp 490 which faces toward the inlet 106 is not shielded. The UV light is emitted through the non-shielded region to shine toward the inner surface 111 of the housing 102 as well as the rear wall 138 of the inlet 104. Nonetheless, an individual is not exposed to undesired UV rays, because the non-shielded region 494 is not viewable from the outlet 106. It should be noted that the angles, locations and numbers of shielded and non-shielded regions discussed in relation to FIG. 11 are examples and are not meant to be limiting. It should also be noted that any other angles, locations and numbers of the shielded and non-shielded regions are contemplated.

It is also contemplated that the integrally shielded lamp 290 is able to be used in other air movement devices not specifically mentioned herein. For example, the integrally shielded lamp 290 is able to be utilized in an electrostatic precipitator system described in the U.S. patent application Ser. No. 10/774,759 which is incorporated by reference above. In addition, the values provided above for the angles and arc-lengths of the shielded and non-shielded regions are examples and should not be limited thereto. Thus, other angles and arc-lengths of the shielded and non-shielded regions are contemplated.

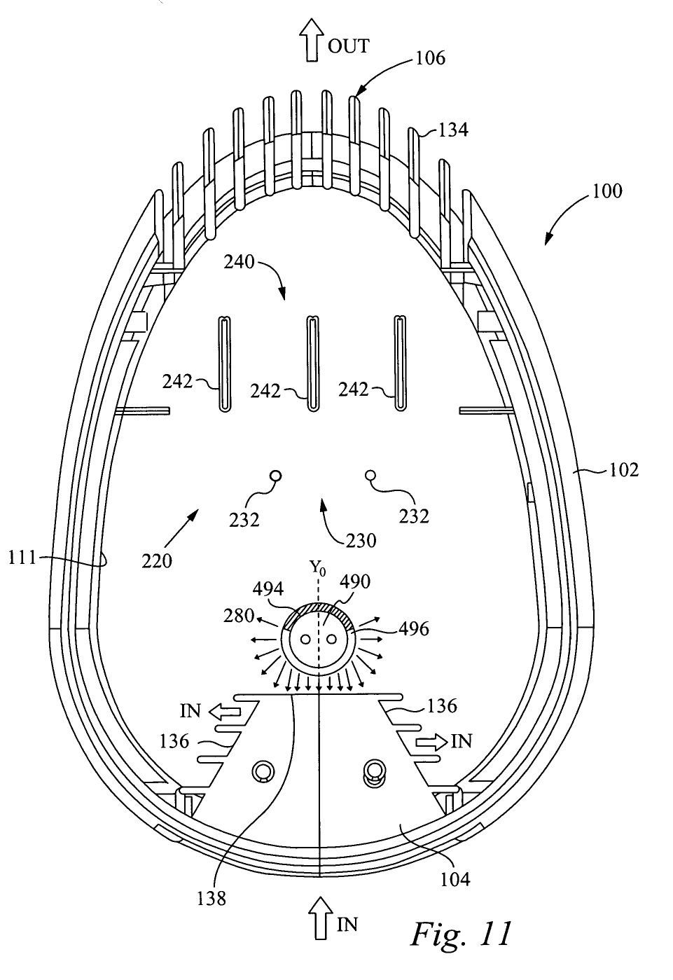

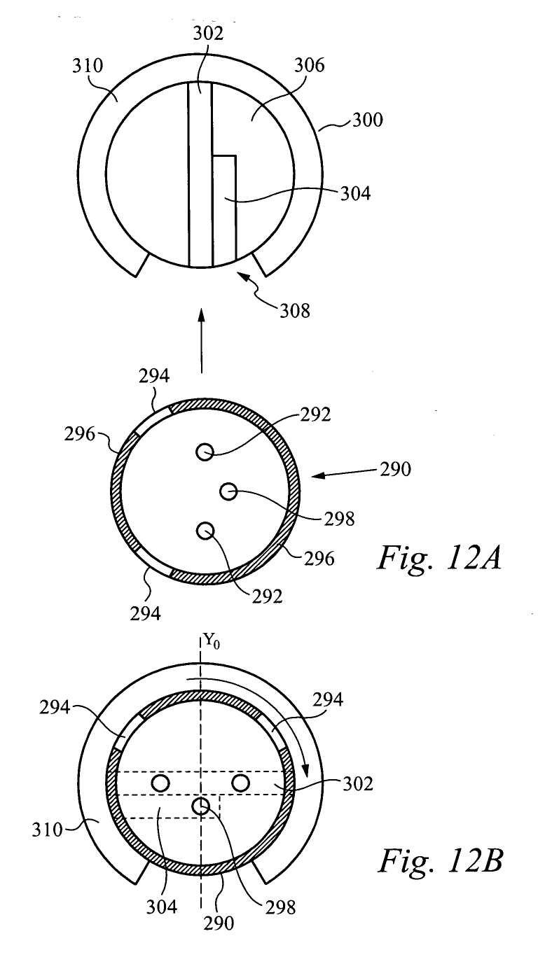

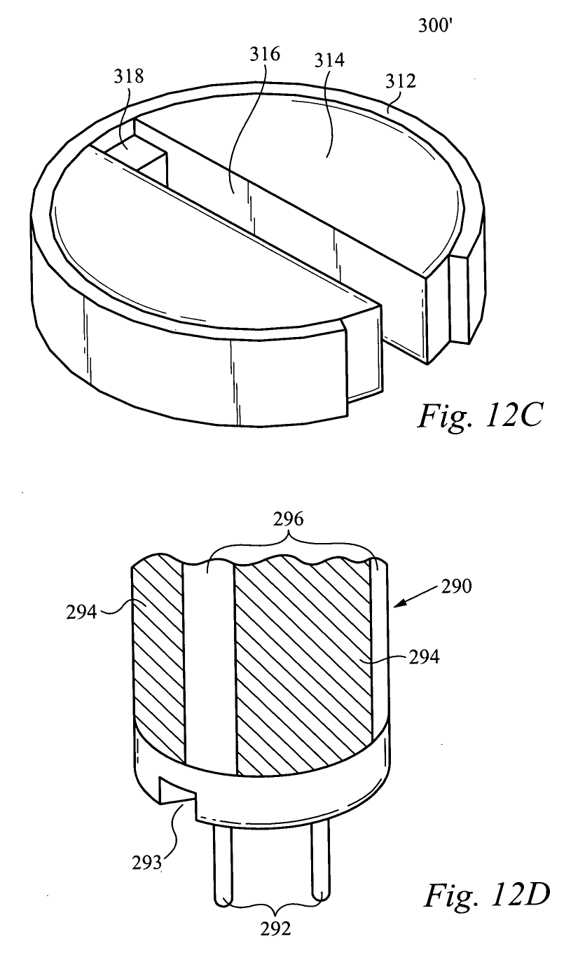

As stated above, the integrally shielded lamp 290 has shielded and non-shielded regions which are to be properly oriented within the housing 102 to prevent undesired UV rays from being directed at the inlet 104 and outlet 106. FIGS. 12A and 12B illustrate plan views of the lamp 290 and receptacle 300 in accordance with one embodiment. As stated above, the integrally shielded lamp 290 couples to a lamp holding receptacle 300, whereby the lamp 290 is selectively removable from the receptacle 300. Preferably, the system 100 includes two receptacles 300, each receptacle to engage an end of the lamp 290. It is preferred that the lamp 290 and/or receptacle 300 be designed such that the lamp 290 can be engaged to the receptacle 300 in only one manner. This ensures that the lamp 290 is oriented properly within the housing 102.

As shown in FIG. 12A, the receptacle housing 300 includes an outer receptacle 310 and an inner receptacle 306 positioned within the outer receptacle 310. The outer receptacle 310 is stationary and mounted to the interior of the housing 102, whereas the inner receptacle 306 is preferably rotatable about its center in the outer receptacle 310. In one embodiment, the inner receptacle 306 is rotated clockwise to a locked position (FIG. 12B). In contrast, the inner receptacle 306 is rotated counterclockwise to be in an unlocked position (FIG. 12A). The lamp 290 is insertable and removable from the receptacle housing 300 through the opening 308 in the outer receptacle 310.

The lamp 290 in FIG. 12A includes the two pins 292 as well as an additional third pin 298 which extends from the end of the lamp 290. Although the terminal pins 292 are aligned along the center at the end of the lamp 290, the third pin 298 is preferably slightly off-center and adjacent to the terminal pins 292. The inner receptacle 306 includes a first recess 302, which receives the two pins 292 as well as a second recess 304 which is slightly off-center to simultaneously receive the off-center third pin 298 of the lamp 290. The offset second recess 304 forces the lamp 290 to be properly inserted in the housing, thereby ensuring that the user properly orients the lamp 290 when engaging the lamp 290 to the receptacle housing 300. Upon properly inserting the pins 292, 298 into their respective recesses 302, 304, the lamp 290 is able to be rotated clockwise approximately 90 degrees to lock the lamp 290 as shown in FIG. 12B. As shown in FIG. 12B, the integrally shielded lamp 290 is oriented in the manner as in FIG. 9 when in the locked position. It is preferred that the pins 292 come into electrical connect with the voltage source when in the secured position shown in FIG. 12B. Removal of the lamp 290 is performed in the opposite manner as that described above. It is preferred that only one of the opposed receptacles 300 includes the second recess 304 to ensure that the lamp 290 is not inserted upside down. However, it is noted that both receptacles 300 can have the design described in FIGS. 12A and 12B.

It should be noted that the above is only one example of how the lamp 290 and receptacle housing 300 are configured and is not to be limited thereto. For example, FIG. 12C illustrates another embodiment of the receptacle housing 300′, whereby the housing 300′ includes the outer receptacle 312 and the rotatable inner receptacle 314. The receptacle housing 300′ is configured to receive the lamp 290′ shown in FIG. 12D. The lamp 290′ in FIG. 12D includes a recess 293 in line with the pins 292 on only one side of the lamp 290′. In the embodiment shown in FIG. 12C, the inner receptacle 314 includes one recess 316 which receives the two pins 292 of the lamp 290′. Within the recess 316 is also a protrusion 318 which serves to mate with the recess 293 (FIG. 12D) of the lamp 290 when the detent 293 end of the lamp 290 is inserted first into the receptacle 300′. For instance, if the non-detent side of the end of the lamp 290 is inserted into the receptacle first, the lamp 290′ will not be able to be completely inserted into the receptacle 300. It is within the scope of the present invention that the present invention utilizes any alternative design to ensure that the lamp 290 operates in the system 100 in the proper orientation such that UV light directly emitted from the lamp 290 does not exit nor is viewed through the inlet and/or outlet grills 104, 106.

FIG. 13 illustrates a perspective view of the front grill with trailing electrodes thereon in accordance with one embodiment of the present invention. As shown in FIG. 13, the trailing electrodes 222 are coupled to an inner surface of the exhaust grill 106. This arrangement allows the user to clean the trailing electrodes 222 from the housing 102 by simply removing the exhaust grill 106. Additionally, placement of the trailing electrodes 222 along the inner surface of the exhaust grill 106 allows the trailing electrodes 222 to emit ions directly out of the system 100 with the least amount of airflow resistance. More details regarding cleaning of the trailing electrodes 222 are described in U.S. Patent Application No. 60/590,735 which is incorporated by reference above.

The operation of replacing the germicidal lamp 290 and cleaning the electrodes of the present system 100 will now be discussed. In one embodiment, the inlet grill 104 is first removed from the housing 102. This is done by lifting the inlet grill 104 vertically and then pulling the grill 104 horizontally away from the housing 102, as discussed above in relation to FIG. 7. Additionally, the exhaust grill 106 is removable from the housing 102 in the same manner. In one embodiment, once the inlet grill 104 is removed from the housing 102, the germicidal lamp 290 is exposed. The user is able to remove the germicidal lamp 290 by preferably twisting the lamp in predetermined direction to unlock the lamp 290 from the lamp receptacle 300. Once unlocked, the, user preferably pulls the lamp 290 laterally outward from within the housing 102. The user is then able to couple a replacement lamp 290 to the housing 102 by inserting the lamp 290 into the receptacle 300 in the correct manner discussed above. Upon locking the lamp 290 within the housing 102, the inlet grill 104 is preferably coupled to the housing 102 in a manner opposite of the grill 104 removal process.

In one embodiment, the user is also able to clean the trailing electrodes 222 on the interior of the grill 106 (FIG. 13). In one embodiment, the user is able to clean the collector and driver electrodes 242, 246 while the electrodes 242, 246 are positioned within the housing 102. In another embodiment, the user is able to pull the collector electrodes 242 telescopically out through an aperture 126 in the top end 124 of the housing 106 as shown in FIG. 6. In one embodiment, the driver electrodes 246 are removed from the housing 102 along with the collector electrodes 242. In another embodiment, the driver electrodes are laterally removable from the housing, either along with removal of the exhaust grill 106 or independently of the removal of the exhaust grill 106. Upon removing the collector and driver electrodes 242, 246, the user is preferably able to clean the electrodes 242, 246 by wiping them with a cloth. Once the collector and driver electrodes 242, 246 are cleaned, the user then inserts the collector and driver electrodes 242, 246 back into the housing 102 in a manner opposite of the removal of the electrodes 242, 246. More detail regarding the insertion and removal of the driver electrodes and collector electrodes are discussed in the 60/590,688 and 60/590,960 applications, which are incorporated by reference above.

The foregoing description of the above embodiments of the present invention has been provided for the purposes of illustration and description. It is not intended to be exhaustive or to limit the invention to the precise forms disclosed. Many modifications and variations will be apparent to one of ordinary skill in the relevant arts. The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalence.

What is claimed is:

1. A germicidal lamp adapted to neutralize particles comprising:
a. a lamp body; and
b. a shield integrally configured with the lamp body, the shield to selectively block UV light emitted directly from the germicidal lamp.

2. The lamp of claim 1 wherein the shield is disposed on an inner surface of the lamp body.

3. The lamp of claim 1 wherein the shield is disposed on an outer surface of the lamp body.

4. The lamp of claim 1 wherein the lamp body has an inner surface and an outer surface, the shield being disposed between the inner and outer surfaces of the lamp body.

5. The lamp of claim 1 wherein the germicidal lamp further comprises a shielded region and a non-shielded region, wherein UV light emitted by the germicidal lamp is emitted only through the non-shielded region.

6. The lamp of claim 5 wherein the germicidal lamp is adapted to be oriented within a housing having an inlet and an outlet such that the shielded region faces the inlet and the outlet.

7. The lamp of claim 1 wherein the germicidal lamp is adapted to be oriented within a housing having an inlet and an outlet, wherein the UV light directly emitted from the germicidal lamp cannot be viewed from at least one of the inlet and the outlet.

8. The lamp of claim 1 wherein the shield further comprises titanium dioxide.

9. The lamp of claim 1 further comprising a non-shielded region, wherein UV light emitted by the germicidal lamp passes through the non-shielded region.

10. The lamp of claim 5 wherein the germicidal lamp is adapted to be oriented within a housing having an interior surface, wherein UV light passing through the non-shielded region is directed toward the interior surface of the housing.

11. The lamp of claim 1 further comprising a reflective material disposed on an interior surface of the lamp body.

12. The lamp of claim 1 further comprising a reflective material disposed on an interior surface of the lamp body, wherein the reflective material intensifies UV light emitted through the non-shielded region.

13. The lamp of claim 1 wherein the germicidal lamp is adapted to be removably coupled to a receptacle housing within a housing, the receptacle housing adapted to engage the germicidal lamp only when the germicidal lamp is in a predetermined orientation with respect to the receptacle housing.

14. The lamp of claim 13 further comprising a timing circuit coupled to the receptacle housing, the timing circuit configured to notify when to replace the germicidal lamp.

15. The lamp of claim 1 further comprising a first shielded region on the lamp body extending between a first end and a second end, the first end at substantially 30 degrees with respect to a Y₀ axis and the second end at substantially 330 degrees with respect to the Y₀ axis.

16. The lamp of claim 15 further comprising a second shielded region on the lamp body extending between a third end and a fourth end, the third end at substantially 80 degrees with respect to the Y₀ axis and the fourth end at substantially 280 degrees with respect to the Y₀ axis.

17. The lamp of claim 1 further comprising:
a. a first shielded region on the lamp body extending between a first end and a second end, wherein the first and second ends are at predetermined angles with respect to a Y₀ axis;
b. a second shielded region on the lamp body extending between a third end and a fourth end, wherein the third and fourth ends are at predetermined angles with respect to the Y₀ axis; and
c. a first non shielded region extending between the first end and the third end.

18. The lamp of claim 1 further comprising:
a. a first shielded region on the lamp body extending between a first end and a second end, wherein the first and second ends are at predetermined angles with respect to a Y₀ axis;
b. a second shielded region on the lamp body extending between a third end and a fourth end, wherein the third and fourth ends are at predetermined angles with respect to the Y₀ axis;
c. a first non-shielded region on the lamp body extending between the first end and the third end; and
d. a second non-shielded region on the lamp body extending between the second end and the fourth end.

19. A germicidal lamp adapted to neutralize particles comprising:
a. a lamp body; and
b. a shield integral with the lamp body, the shield to selectively allow UV light produced from the germicidal lamp to emit through the lamp body.

20. The lamp of claim 19 wherein the shield is disposed on an inner surface of the lamp body.

21. The lamp of claim 19 wherein the shield is disposed on an outer surface of the lamp body.

22. The lamp of claim 19 wherein the lamp body has an inner surface and an outer surface, the shield being disposed between the inner and outer surfaces of the lamp body.

23. The lamp of claim 19 wherein the germicidal lamp further comprises a shielded region and a non-shielded region, wherein UV light emitted by the germicidal lamp is emitted only through the non-shielded region.

24. The lamp of claim 23 wherein the germicidal lamp is adapted to be oriented within a housing having an inlet and an outlet such that the shielded region faces the inlet and the outlet.

25. The lamp of claim 23 wherein the germicidal lamp is adapted to be oriented within a housing having an inlet and an outlet, wherein the shielded region faces the inlet and the outlet and the non-shielded region faces an interior wall of the housing.

26. The lamp of claim 19 wherein the shield further comprises titanium dioxide.

27. The lamp of claim 23 wherein the germicidal lamp is adapted to be oriented within a housing having an interior surface, wherein the UV light passing through the non-shielded region is directed toward the interior surface of the housing.

28. The lamp of claim 19 further comprising a reflective material disposed on an interior surface of the lamp body.

29. The lamp of claim 19 wherein the germicidal lamp is adapted to be removably coupled to a receptacle housing within a housing, wherein the receptacle housing engages the germicidal lamp only when the germicidal lamp is positioned in a predetermined orientation with respect to the receptacle housing.

30. The lamp of claim 19 further comprising a first shielded region on the lamp body extending between a first end and a second end, the first end at substantially 30 degrees with respect to a Y₀ axis and the second end at substantially 330 degrees with respect to the Y₀ axis.

31. The lamp of claim 32 further comprising a second shielded region on the lamp body extending between a third end and a fourth end, the third end at substantially 80 degrees with respect to the Y₀ axis and the fourth end at substantially 280 degrees with respect to the Y₀ axis.

32. The lamp of claim 19 further comprising:
a. a first shielded region on the lamp body extending between a first end and a second end, wherein the first and second ends are at predetermined angles with respect to a Y₀ axis;
b. a second shielded region on the lamp body extending between a third end and a fourth end, wherein the third and fourth ends are at predetermined angles with respect to the Y₀ axis; and
c. a first non shielded region extending between the first end and the third end.

33. The lamp of claim 19 further comprising:
a. a first shielded region on the lamp body extending between a first end and a second end, wherein the first and second ends are at predetermined angles with respect to a Y₀ axis;
b. a second shielded region on the lamp body extending between a third end and a fourth end, wherein the third and fourth ends are at predetermined angles with respect to the Y₀ axis;
c. a first non-shielded region on the lamp body extending between the first end and the third end; and
d. a second non-shielded region on the lamp body extending between the second end and the fourth end.

34. A lamp system comprising:
a. a removable germicidal lamp having an integral shield to selectively block UV light emitted directly from the germicidal lamp; and
b. a lamp receptacle adapted to engage the germicidal lamp, wherein the lamp receptacle engages the germicidal lamp only when the germicidal lamp is in a predetermined orientation with respect to the lamp receptacle.

35. The system of claim 36 further comprising a timing circuit coupled to the lamp receptacle, wherein the timing circuit is configured to notify when to replace the germicidal lamp.
